(19) United States
(12) Patent Application Publication
(10) Pub. No.: US 2004/0146502 A1
(43) Pub. Date: Jul. 29, 2004

(54) Use of organic compounds

(76) Inventors: Elisabeth Latour, Bartenheim (FR); George N. Lambrou, Strasbourg (FR)

Correspondence Address: NOVARTIS, CORPORATE INTELLECTUAL PROPERTY, ONE HEALTH PLAZA 430/2, EAST HANOVER, NJ, 07936-1080, US

(21) Appl. No.: 10/476,509

(22) PCT Filed: May 2, 2002
(86) PCT No.: PCT/EP02/04820

(30) Foreign Application Priority Data
May 3, 2001 (EP) 01110752.1

(51) Int. Cl. A61K 39/395

(52) U.S. Cl. 424/131.1

(57) ABSTRACT

The present invention relates to the use of an anti-IgE antibody in the preparation of a topical ophthalmic composition for the treatment of an ocular allergic disorder and a method to treat ocular allergic disorders in a patient in need therefore, which method comprises the topical administration of an opthalmic composition comprising an anti-IgE antibody.

The present invention relates to the use of an anti-IgE antibody and in particular to the use of an antibody comprising a sequence selected from E25, E26 and mixtures thereof in the preparation of a topical ophthalmic composition for the treatment of an ocular disorder.

A series of improved anti-IgE antibodies including antibodies comprising a sequence selected from E25, E26 and mixtures thereof are described in full detail in WO 99/01556. Said prior art describes the use in the treatment of IgE-mediated disorders, which is in particular characterized by the overproduction and/or hypersensitivity to the immunoglobulin IgE. However, prior art is silent with respect to specific ocular allergic disorders.

It was surprisingly found that an ophthalmic composition comprising an anti-IgE antibody is useful in the topical treatment of an ocular allergic disorder and in the preparation of a topical ophthalmic composition in the treatment of an ocular disorder. The addressed ophthalmic compositions exhibit an excellent ocular tolerability, a short onset of action, a long duration of action and an excellent clinical efficacy.

The clinical effect, such as ocular tolerability and efficacy of the addressed ophthalmic compositions is tested pre-clinically, and for example in rabbit or guinea pig eye.

Given the large size of the addressed antibodies, ocular penetration and consequently ocular efficacy is highly unexpected by the skilled man in the art.

Throughout this Invention, the term topical refers in particular to the topical ocular environment, which contains tear fluid.

In one aspect the present invention therefore relates to the use of an anti-IgE antibody, in particular an antibody comprising a sequence selected from E25, E26 and mixtures thereof, in the preparation of a topical ophthalmic composition for the treatment of an ocular allergic disorder.

An especially preferred antibody is a sequence selected from E25.

The anti-IgE antibodies are described in the prior art, and in greater detail in the International applications WO 93/04173 and WO 99/01556. WO 99/01556 specifically describes E25 in FIG. 12, and in the sequences ID-No. 13-14. Antibody molecules comprising a E26 sequence are described in WO 99/01556 and are selected from the group of F(ab) fragment (Sequence ID Nos. 19-20), sFv fragment (Sequence ID No. 22) and F(ab)′₂ fragment (Sequence Nos. 24-25), in accordance to FIGS. 12-15. Therefore, within this invention, the terms E25 and E26 shall be construed accordingly.

In another aspect the invention relates to the method to treat ocular allergic disorders in a patient in need therefore, which method comprises the topical administration of an ophthalmic composition comprising an anti-IgE antibody and in particular comprising a sequence selected from E25, E26 and mixtures thereof.

Within this invention, the term ocular allergy shall refer typically but not exclusively to five different clinical entities, namely:

Seasonal (hay fever) and perennial allergic conjunctivitis

Vernal keratoconjunctivitis

Atopic keratoconjunctivitis

Giant-papillary conjunctivitis, and

Contact ocular allergy.

More preferably the term ocular allergy shall refer to

Seasonal (hay fever) and perennial allergic conjunctivitis

Vernal keratoconjunctivitis

Atopic keratoconjunctivitis, and

Giant-papillary conjunctivitis.

Even stronger preferred the term ocular allergy shall refer to

Seasonal (hay fever) and perennial allergic conjunctivitis

Vernal keratoconjunctivitis, and

Atopic keratoconjunctivitis.

Most preferably the term ocular allergy shall refer to

Seasonal (hay fever) and perennial allergic conjunctivitis, and

Atopic keratoconjunctivitis.

Buffers, tonicity enhancing agents and preservatives may be used in an ophthalmic composition of the present invention as well.

Examples of buffer substances are acetate, ascorbate, borate, hydrogen carbonate/carbonate, citrate, gluconate, lactate, phosphate, propionate and TRIS (tromethamine)buffers. Tromethamine and borate buffer are preferred buffers. The amount of buffer substance added is, for example, that necessary to ensure and maintain a physiologically tolerable pH range. The pH range is typically in the range of from 5 to 9, preferably from 6 to 8.5 and more preferably from 6.5 to 8.2.

Tonicity enhancing agents are, for example, ionic compounds, such as alkali metal or alkaline earth metal halides, such as, for example, CaCl₂, KBr, KCl, LiCl, NaI, NaBr or NaCl, or boric acid. Non-ionic tonicity enhancing agents are, for example, urea, glycerol, sorbitol, mannitol, propylene glycol, or dextrose. Typically, sufficient tonicity enhancing agent is added to impart to the ready-for-use ophthalmic composition an osmolality of approximately from 50 to 1000 mOsmol, preferred from 100 to 400 mOsmol, more preferred from 200 to 400 mOsmol and even more preferred from 250 to 350 mOsmol.

Examples of preservatives are quaternary ammonium salts, such as benzalkonium chloride, parabens, such as, for example, methylparaben or propylparaben, alcohols, such as, for example, chlorobutanol, benzyl alcohol or phenyl ethanol, guanidine derivatives, such as, for example, chlorhexidine or polyhexamethylene biguanide, or sorbic acid. Preferred preservatives are quaternary ammonium salts and parabens. Where appropriate, a sufficient amount of preservative is added to the ophthalmic composition to ensure protection against secondary contaminations during use caused by bacteria, fungi and the like.

1. Use of an anti-IgE antibody in the preparation of a topical ophthalmic composition for the treatment of an ocular allergic disorder.

2. Use of claim 1, wherein said antibody comprises a sequence selected from E25, E26 and mixtures thereof.

3. Use of claim 1, wherein said antibody comprises a sequence selected from E25.

4. Use of claim 1, wherein said allergic disorder is selected from the group consisting of:
Seasonal (hay fever) and perennial allergic conjunctivitis
Vernal keratoconjunctivitis
Atopic keratoconjunctivitis
Giant-papillary conjunctivitis, and
Contact ocular allergy.

5. Use of claim 4, wherein said allergic disorder is selected from
Seasonal (hay fever) and perennial allergic conjunctivitis
Vernal keratoconjunctivitis
Atopic keratoconjunctivitis, and
Giant-papillary conjunctivitis.

6. Use of claim 4, wherein said allergic disorder is selected from
Seasonal (hay fever) and perennial allergic conjunctivitis
Vernal keratoconjunctivitis, and
Atopic keratoconjunctivitis.

7. Use of claim 4, wherein said allergic disorder is selected from
Seasonal (hay fever) and perennial allergic conjunctivitis, and
Atopic keratoconjunctivitis.

8. Method to treat an ocular allergic disorder in a patient suffering from said allergic disorder, which method comprises the topical administration of an ophthalmic composition comprising an anti-IgE antibody.

9. Method of claim 8, wherein said antibody comprises a sequence selected from E25, E26 and mixtures thereof.

10. Method of claim 8, wherein said allergic disorder is selected from:
Seasonal (hay fever) and perennial allergic conjunctivitis
Vernal keratoconjunctivitis
Atopic keratoconjunctivitis
Giant-papillary conjunctivitis, and
Contact ocular allergy.
